(19) United States
(12) Patent Application Publication
(10) Pub. No.: US 2010/0049142 A1
(43) Pub. Date: Feb. 25, 2010

(54) ATTACHMENT FOR A PARENTERAL DEVICE

(75) Inventor: Douglas Arthur Sims, Western Australia (AU)

Correspondence Address: MERCHANT & GOULD PC, P.O. BOX 2903, MINNEAPOLIS, MN, 55402-0903, US

(73) Assignee: Eastland Medical Systems Ltd., Perth (AU)

(21) Appl. No.: 12/300,977

(22) PCT Filed: May 14, 2007
(86) PCT No.: PCT/AU2007/000647

§ 371 Date: Sep. 30, 2009

(30) Foreign Application Priority Data
May 17, 2006 (AU) 2006902632

(51) Int. Cl. A61M 5/32

(52) U.S. Cl. 604/196

(57) ABSTRACT

An attachment for a parenteral device wherein the parenteral device comprises a body defining space and a duct having an opening at its outer end, the duct in use is intended to convey parenteral fluid to or from the space, said attachment comprising a housing adapted to be removably engageable with the body and to be supported by the body, the housing having an engagement portion (13) having a flow path (29) which is to be in communication with the opening and the duct when the engagement portion is in said sealing engagement with the body the housing further comprising an elongate portion (11) slidably supporting a hollow needle (15) wherein the needle has a free end (17) which is open and a handle (20) mounted to the other end, the other end of the needle being closed, the needle provided with a second opening (19) formed through the wall of the needle intermediate of the ends of the needle, the needle being slidably accommodated within the elongate housing to be movable from or to a stored position at which the free end is sealingly accommodated within the housing, to or from an extended position at which the free end extends beyond the housing, the needle being further movable to a retracted position at which the free end is located in a non-sealing manner within the housing, the housing having a sub-chamber which is open to the flow path whereby the second opening is located within the sub-chamber on the needle being moved to the extended position.

### FIELD OF THE INVENTION

This invention relates to an attachment which can be utilised with syringes and other parenteral devices.

### BACKGROUND ART

The invention relates the field of retractable needle parenteral devices in which a needle is slidably supported in the body of the device to movable between a stored position at which the needle is fully accommodated within the body to be inaccessible and an extended position at which the needle extends beyond the body whereby on the needle being moved to the extended position, the interior of the needle is in communication with a chamber and/or duct whereby fluid can be transferred through the needle to or from the chamber.

In particular the invention relates to an attachment which can be utilised with a syringe and which has a retractable needle and comprises a development of the attachment which is the subject of Australian patent 659246.

Throughout the specification and claims the term “syringe” shall be taken to include parenteral devices which can be used to effect the conveyance of a parenteral fluids which are to be introduced into or drawn from the body of patient and shall include within its scope a syringe, a cannular, a hypodermic needle, an intravenous infusion line and like parenteral devices.

The discussion throughout this specification, of the background and prior art to the invention is intended only to facilitate an understanding of the present invention. It should be appreciated that the discussion is not an acknowledgement or admission that any of the material referred to was part of the common general knowledge in Australia and the world as at the priority date of the application.

It is an object of this invention to provide an attachment which can be used with a conventional syringe and which will enable a needle to be mounted to the syringe whereby the needle of the attachment can be extended or retracted in a controlled manner as required by the user. This arrangement enables the attachment to be used with any form of conventional syringe.

### DISCLOSURE OF THE INVENTION

Throughout the specification and claims, unless the context requires otherwise, the word “comprise” or variations such as “comprises” or “comprising”, will be understood to imply the inclusion of a stated integer or group of integers but not the exclusion of any other integer or group of integers.

Accordingly, the invention resides in an attachment for a parenteral device wherein the parenteral device comprises a body defining space and a duct having an opening at its outer end, the duct in use is intended to convey parenteral fluid to or from the space, said attachment comprising a housing adapted to be removably engageable with the body and to be supported by the body, the housing having an engagement portion having a flow path which is to be in communication with the opening and the duct when the engagement portion is in said sealing engagement with the body the housing further comprising an elongate portion slidably supporting a hollow needle wherein the needle has a free end which is open and a handle mounted to the other end, the other end of the needle being closed, the needle provided with a second opening formed through the wall of the needle intermediate of the ends of the needle, the needle being slidably accommodated within the elongate housing to be movable from or to a stored position at which the free end is sealingly accommodated within the housing to or from an extended position at which the free end extends beyond the housing, the needle being further movable to a retracted position at which the free end is located in a non-sealing manner within the housing, the housing having a sub-chamber which is open to the flow path whereby the second opening is located within the sub-chamber on the needle being moved to the extended position.

According to a preferred feature of the invention the dimensions of the sub-chamber enable open communication between the interior of the needle and the sub-chamber when the opening is located within the irrespective of the angular orientation of the needle relative to the sub-chamber.

According to a preferred feature of the invention one end of the housing is formed with a bore through which the needle is slidably received and the sub-chamber is located intermediate along the length of the bore, wherein the bore is dimensioned such that the needle is slidably and sealing engaged by the inner face of the bore to each side of the sub-chamber. According to a preferred feature of the invention the sub-chamber has dimensions greater than the bore. According to a preferred feature of the invention the free end is located within the bore but outward of the sub-chamber when the needle is in the stored position and the free end is located inward of the bore when the needle is located in the retracted position.

According to a preferred feature of the invention the duct comprises a tubular spigot extending from the parenteral device.

According to a preferred feature of the invention the spigot has a configuration of a “Luer” fitting. According to a preferred feature of the invention the “Luer” fitting is associated with a “Luer Lock”

According to a preferred feature of the invention the engagement portion includes a socket which is adapted to sealingly engage the body to surround the opening, said socket being located to one side of the elongate portion, said flow path extending between the socket and the sub-chamber. According to a preferred feature of the invention the flow path comprises a first longitudinal passage formed through the engagement portion said longitudinal passage connecting the socket with the sub-chamber. According to a preferred feature of the invention the sub chamber is a part of the longitudinal passage and is coaxial therewith and the longitudinal passage intersects the bore in the region of the sub-chamber. According to a preferred feature of the invention the sub chamber has a diameter greater than the longitudinal passage diameter greater than the diameter of the bore. According to an alternative preferred feature of the invention the sub chamber has a diameter substantially equal to the diameter of the longitudinal passage. According to an alternative preferred feature of the invention the longitudinal passage including the sub chamber has tapered internal cross-section which is convergent in the direction away from the bore. According to a preferred feature of the invention the diameter of the sub-chamber is greater than the diameter of the bore. According to a preferred feature of the invention the longitudinal passage is substantially perpendicular to the bore. According to a preferred feature of the invention the longitudinal passage opens to the exterior of the engagement portion in opposed relation to the socket and the outer end of the longitudinal passage is closed. According to a preferred feature of the invention the longitudinal passage is closed subsequent to the formation of the longitudinal passage.

According to a preferred feature of the invention the engagement portion is formed as a separate element from the elongate portion.

According to a preferred feature of the invention the handle comprises a first element which is slidably received in the elongate body and which closes the other end of the needle, the first element supporting a protuberance which extends from the elongate body and is engageable by an operator to effect said slidable movement of the needle. According to a preferred feature of the invention the handle supports a pawl which is engageable with one of a set of longitudinally spaced recesses in the elongate portion to retain the needle in the stored, extended and retracted position. According to a preferred feature of the invention the pawl is resiliently supported from the first element such that it is biased laterally with respect to the direction of movement of the needle to become engaged with one of said recesses on the pawl moving into alignment with a recess. According to a preferred feature of the invention an increased biasing is applied to the pawl when in engagement with the recess at which the needle is in the retracted position.

According to a preferred feature of the invention the parenteral device comprises a syringe and wherein when the syringe has the attachment engaged therewith the central axes of the elongate potion and the syringe are substantially parallel and the elongate portion lies closely adjacent to the syringe.

The invention will be more fully understood in light of the following description of several specific embodiments.

### BRIEF DESCRIPTION OF THE DRAWINGS

The description is made with reference to the accompanying drawings, of which;

FIG. 1 is a sectional view of an attachment for a syringe according to the first embodiment showing the needle in the stored position;

FIG. 2 is an isometric view of an attachment for a syringe according to the first embodiment showing the needle in the stored position and in which the engagement portion has been sectioned;

FIG. 3 is a sectional elevation of the attachment of FIG. 1 showing the needle in an extended position;

FIG. 4 is an isometric view of an attachment for a syringe according to the first embodiment showing the needle in the “extended position”;

FIG. 5 is sectional elevation of the attachment of FIG. 1 showing the needle in the retracted position; and

FIG. 6 is a sectional view of an attachment for a syringe according to the second embodiment showing the needle in the stored position and with the attachment mounted to a syringe.

### DETAILED DESCRIPTION OF SPECIFIC EMBODIMENTS

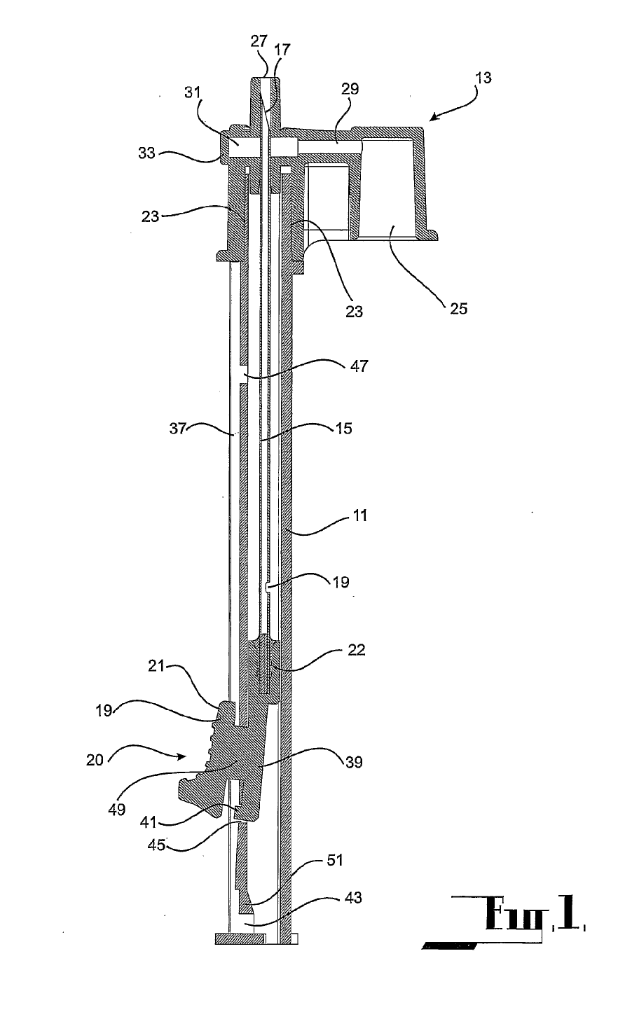

The first embodiment of the invention as shown at FIGS. 1 to 5 of the accompanying drawings comprises a development of the invention which is disclosed in Australian patent 659246. In manufacturing the product according to the invention of the abovementioned patent, difficulties have been encountered in regard to the number of components that are required and in the formation of each of the components.

The present embodiment comprises an attachment which can be utilised with conventional syringe (not shown). The conventional syringe comprises a body which is of a generally tubular form and provides space which slidably accommodates a plunger to define a chamber whereby the volume of the chamber can be varied with movement of the plunger within the space. The end of body opposite the plunger is closed and is formed with a tubular spigot which has duct which opens at one end into the chamber and has an opening at the outer end of the boss. The spigot is generally referred to as a Luer fitting or spigot.

The function of the embodiment is to provide an attachment means whereby a needle can be mounted to the syringe and whereby the needle is movable from a stored position at which it is fully protected, an extended position at which the free end is exposed and a retracted position at which the needle is located such that it cannot again be moved to the extended position. It is also a function of the embodiment to provide an attachment whereby the needle is separable from the syringe and can be disposed of separately from the syringe.

The attachment according to the first embodiment comprises a housing which consists of an elongate portion 11 which is hollow and which is closed at one end by an engagement portion 13 which is formed separately and is fixed to the one end of the elongate body. The elongate portion slidably accommodates a hollow needle 15. The needle 15 is formed with a free end 17 which is open to allow access to the lumen of the needle. The other end is closed. In the case of the embodiment the other end is closed by a handle element 20 which not only closes the other end of the needle, it is also formed with a manipulation means 21 which extend outwardly from the elongate portion. The handle element 20 is also formed with a slider 22 which sealingly closes the other end of the needle and supports the other end of the needle within the elongate body. The slider 22 is slidably received within the interior of the elongate portion 11 to enable slidable movement of the handle element 20 along the elongate portion and as a result of such movement to cause the needle to move within the elongate portion.

The needle is also formed with an opening 19 which extends through the wall of the needle and is located intermediate of its length but towards the handle 20.

The engagement portion 13 comprises a first socket 23 which is configured to be fixed to the one end of the elongate portion 11 and a second socket 25 which is configured to sealing engage with the tubular spigot (not shown) provided on the syringe. The central axes of the first and second sockets are substantially parallel and are spaced from each other such that when the second socket is sealingly fixed to the tubular spigot of the syringe, the elongate portion 11 lies closely adjacent to and parallel to the main body of the syringe.

The engagement portion 13 is formed with a bore 27 which extend from the base of the first socket 23 to the outer face of the engagement portion 13 and which is coaxial with the interior of the elongate portion but of a reduced diameter. The bore 27 has a diameter corresponding to that of the needle such that a tight and snug engagement is effected between the walls of the needle and the bore 27 when the needle is located within the bore. To facilitate the movement of the needle through the bore, the material of which the engagement potion is formed is a “self-lubricating form” of plastics material which to allows for slidable movement of the needle through the bore whilst maintaining a sealing engagement between the needle and the bore 27.

The engagement portion 13 is further provided with a longitudinal passage 29 which is perpendicular to the bore 27 and intersects the bore intermediate of the length of the bore. As result the needle 15 is required to extend through sub-longitudinal passage 29 when it is fully engaged in the bore. The longitudinal passage 29 extends to the second socket 25 to provide communication between the bore 27 and the second socket 25. In the area of the intersection of the longitudinal a passage with the bore the longitudinal passage is formed provide a sub-chamber 31 which has an increased diameter which is greater than the diameter of the needle.

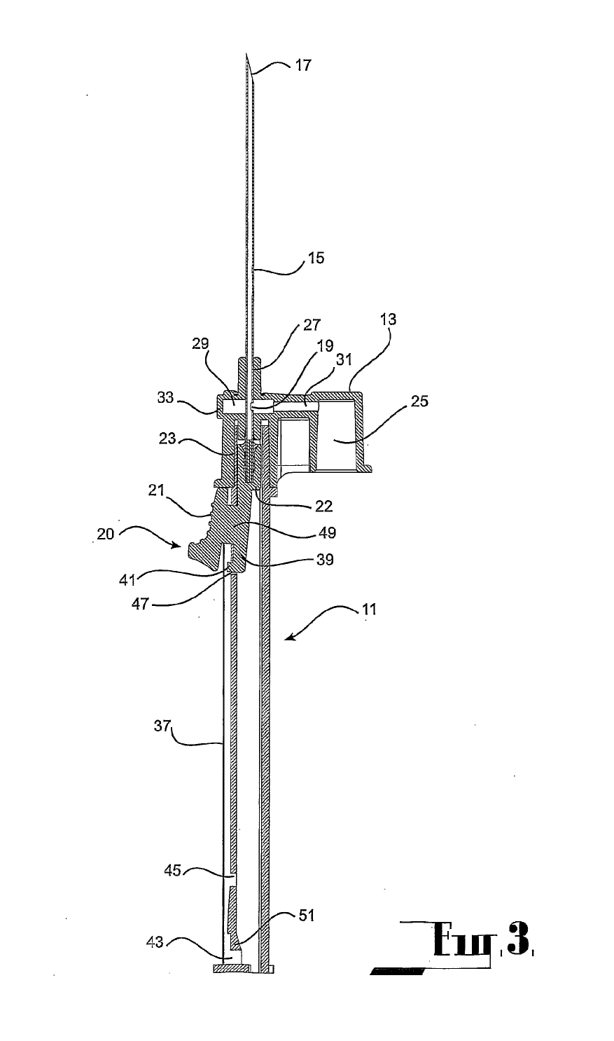

As a result of the fact that the bore is of a greater diameter than the bore, when the opening 19 of the needle is located within the sub chamber 31 (see FIG. 3), the opening 19 in the side wall of the needle is in open communication with interior the sub-chamber irrespective of the angular displacement of the needle relative to the central axis of the sub-chamber.

The longitudinal passage 29 opens into the second socket 25 to provide a flow path between the sub-chamber 31 and the second socket 25 and thus to enable fluid communication between the second socket 25 and the sub-chamber 31. The portion of the longitudinal passage 29 between the sub-chamber 31 and the second socket 25 is formed as a longitudinal extension of the sub-chamber 31 but is of a reduced diameter.

The engagement portion 13 is formed of said plastics material by pressure injection moulding. In forming the longitudinal passage 29 including the sub-chamber 31, a moulding pin is inserted into the mould forming the engagement portion. The moulding pin is inserted into the mould from the opposite side of the mould to the second socket 25. The moulding pin is formed with an outer axial portion and inner axial portion where the outer axial portion is of a reduced diameter compared to the inner axial portion, and where the outer axial portion of the moulding pin forms the extension of the longitudinal passage 27 which opens into the second socket 25 and the inner axial portion of the moulding pin forms the sub-chamber 31. In the forming of the engagement portion and on the introduction of the plastics material into the mould the moulding pin is inserted into the mould to form the longitudinal passage 29 which includes the sub-chamber 29. On the hardening of the plastics material the moulding pin is withdrawn from the mould. The outer end of the longitudinal passage which forms the sub-chamber 31 and which opens to the opposite side from the second socket 25 is subsequently closed by a plug 33.

The elongate portion 11 of the attachment is hollow and is of a generally square cross-section. One side wall of the elongate portion is formed with a slot 35. The one side is also formed at each side a flange 37 which extends for the length of the one side wall and which extends perpendicularly from the one side wall to define a channel along the one side wall whereby the slot 35 is located centrally within the channel and at the base of the channel. The manipulation means 21 of the handle element 20 is slidably received within the channel for movement there along and is connected to the slider 22 which is slidably received within interior of the elongate portion by an extension 39 of the slider which is slidably received within the slot 35. The extension 39 is connected to the manipulation means 21 by a neck portion 49 which has transverse dimensions corresponding to the slot 35. The extension 39 is provided with a transversely directed pawl 41 at its outer end which has transverse dimensions greater than the slot 35 where the pawl is biased to be slidably received over the inner face of the one side wall to each side of the slot 35. The slot 35 is provided with a set of lateral extensions 43, 45 and 47 at spaced intervals along the slot. The lateral extensions have a dimension such that they will receive the pawl 41. The handle element 20 is formed of a suitably resilient plastics material and the configuration of the manipulation means 21 is such that under normal conditions the pawl 41 is biased transversely outwardly from the interior of the elongate body to bear upon the inner face of the one side wall to each side of the slot 35. When the pawl 41 is in alignment with one of the lateral extensions 43, 45 or 47 pawl the biasing which is applied to the pawl 41 will cause it to move into engagement into that lateral extension. The manual inward depression of the manipulation means 21 against the biasing force will cause inward deflection of the extension 99 to cause the pawl 41 to be moved out of engagement with the relevant lateral extension, and as a result the handle is capable of moving along the length of the elongate portion and carrying the needle with it.

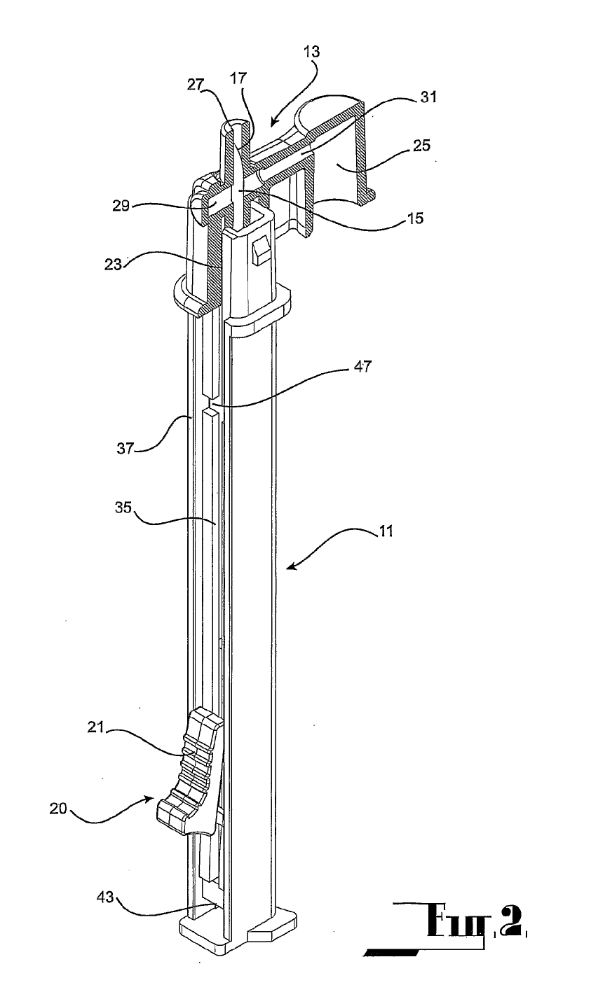

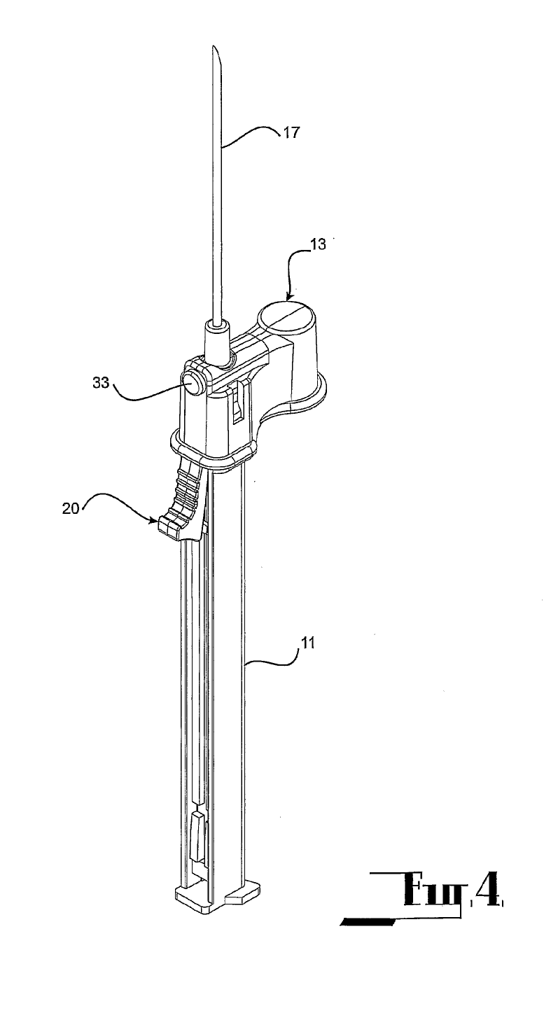

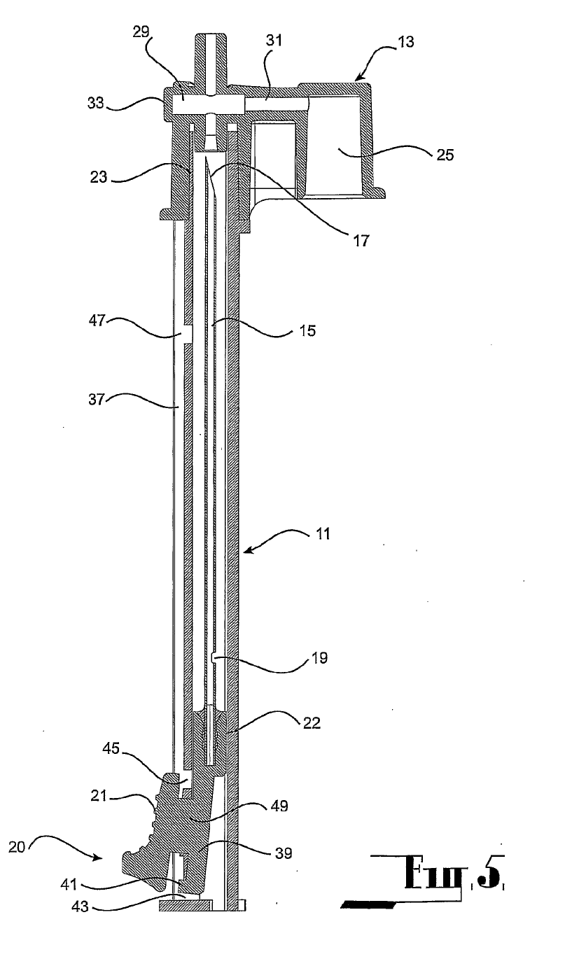

The needle is movable between three positions which comprise a stored position as shown at FIGS. 1 and 2, an extended position as shown at FIGS. 3 and 4 and a retracted position as shown at FIG. 5. Each position is associated with one of the lateral extensions 43, 45 or 47. At each position, the pawl 41 is engaged with a lateral extension corresponding to the position as result of the biasing provided to the pawl 41 by the resilience of the extension 39. Such resilience causes the pawl to engage with the respective lateral extension on it becoming aligned with the lateral extension.

The intermediate lateral extension 45 accommodates the pawl 41 when the needle is located in its stored position as shown at FIGS. 1 and 2 and when at this position such engagement prevents the needle from moving longitudinally within the elongate portion. When the needle is at its extended position as shown at FIGS. 3 and 4 the pawl 41 is located in the lateral extension 47 most adjacent the engagement portion 13. When the needle is its retracted position as shown at FIG. 5, the pawl 41 is located in the end most lateral extension 43.

When in the stored position as shown at FIGS. 1 and 2 the free end 17 of the needle is sealingly located within the bore 27 outward of the sub-chamber 29. As a result of that arrangement any fluid contained within the longitudinal passage 29, sub-chamber 31, second socket 25 and the chamber of the syringe is isolated.

On it becoming necessary to use the needle, the manipulation means 21 is then depressed and the handle is moved longitudinally along the elongate body in the direction of the engagement portion 13 until the pawl 41 becomes engaged with the lateral extension 47 most adjacent the engagement portion 13 at which position the needle is locked into its extended position as shown at FIGS. 3 and 4. At this position the opening 19 provided in the needle is accommodated within the sub-chamber and as a result there is fluid communication between the lumen of the needle, sub-chamber 33, the longitudinal passage 27, the second socket 25 and the interior of the syringe with which the attachment is associated. This will then enable the fluid within the chamber of the syringe to be delivered into the needle and thus into the body of the patient or alternatively fluid from the patient to be drawn into the chamber of the syringe through the needle.

On completion of utilisation of the needle the manipulation means 21 is again depressed to cause the pawl 41 to disengage from the lateral extension 47 most adjacent the engagement portion 13 and as a result the needle 21 can be moved rearward to the stored position as shown at FIG. 1 or 2 or if moved beyond the intermediate lateral extension 45 to the retracted position at which the pawl is engaged in the end most lateral extension 43.

The inner face of the one side wall to each side of the slot immediately adjacent the endmost lateral extension 43 is formed with a ramped portion 51 which will tend to resiliently deflect the extension 39 away from the one side wall as it approaches the endmost lateral extension. 43. As a result when the pawl is located within the endmost lateral extension 43 the needle is retained at that position. To become disengaged form that position the amount of force required to cause disengagement of the pawl 41 from the end most extension 43 is much greater than with the other lateral extensions 45 and 47. In addition when the needle is at the retracted position, the free end 17 of the needle is located inward of the bore 27 of the sealing extension 13. The resultant effect is that depression of the manipulation means to cause the pawl 41 to disengage from the endmost lateral extension 43 will cause the needle to move out of axial alignment with the bore 27 and because of the sharpness of the free end 17 the needle is unlikely to be readily reengaged with the bore 27. Furthermore the initial misalignment of the needle with respect to the bore is rendered more likely because of the influence of the engagement of the pawl 41 with the ramped portion 51 which will serve to cause such misalignment

The first embodiment provides an attachment for a syringe whereby the functions of a retractable needle can be associated with a conventional syringe. In addition, the needle of the attachment is capable of moved between:
a stored position at which the contents of the syringe can be isolated;
an extended position at which the contents of the syringe can be utilised; and
a retracted position at which the attachment can not be re-used.

Furthermore, the attachment enables the needle to be retracted to its stored position from the extended position in order that it can be reused if necessary. These circumstance can arise in situations where the injection or collection procedure has to be interrupted due to environmental conditions and/or difficulties created by movement in the patient or a like situation and the injection or collection procedure can be reinitiated once the circumstances have become stabilised.

The first embodiment also enables the attachment to be utilised to draw a fluid from an ampoule or a like container into a syringe, and once the required contents have been introduced into the syringe, the needle can be retracted to its stored position at which the free end of needle 17 is inaccessible and the contents of the syringe is are isolated. When it becomes necessary to inject the fluid, the needle can be then be moved to its extended position.

The stepped nature of the longitudinal passage 27 by providing the sub-chamber of greater diameter than the needle in the region of the bore only, serves to reduce the volume of the space defined with in the engagement portion between the second socket 25 and the needle and thus the volume of fluid which will be retained by the attachment. This can be particularly relevant in situations where the volume of the dosage to be delivered is small.

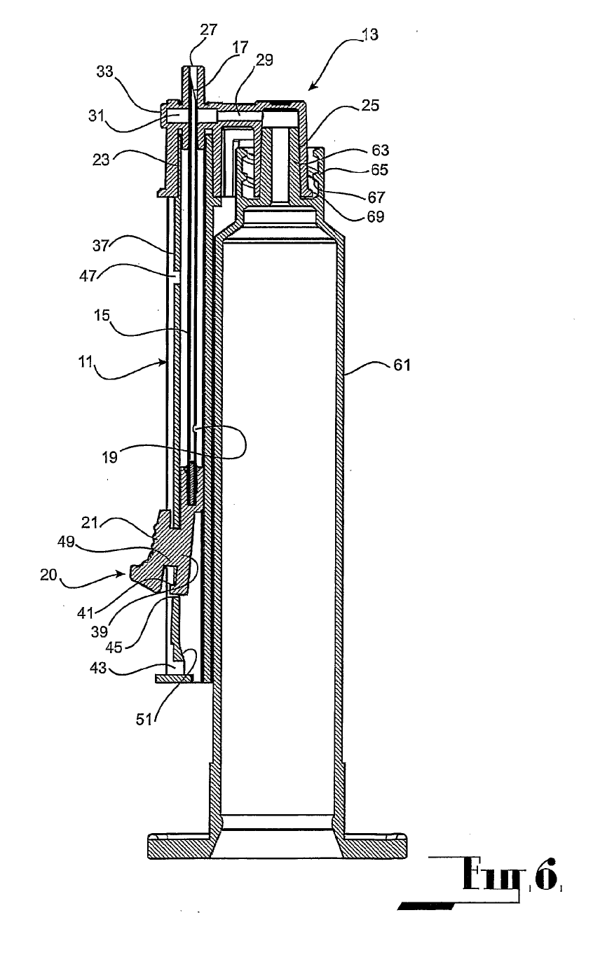

A second embodiment of the invention is shown at FIG. 6 and the same reference numerals are used with elements which correspond to the elements shown in FIGS. 1 to 5. FIG. 6 illustrates the second embodiment mounted to a syringe 61 which has Luer spigot 63 which is sealingly engaged with the second socket 25 of the engagement portion 13. In addition the Luer spigot 63 is associated with an annular shroud 65 which surrounds the spigot and is provided on its inner face with a helical rib 67 which provides a thread-like formation on the inner face of the shroud. In addition the external; edge of the second socket is formed with a protrusion 69 which extends radially from one side of the outer edge of the second socket. The protrusion 69 is dimensioned to be threadably received by the helical rib 67 when the second socket 25 is engaged with the Luer spigot 61. In so doing the when the second socket is initially engaged with the Luer spigot it is caused to rotate around the syringe to allow the second socket 25 to become threadably engaged with the shroud 65. As a result the engagement portion 13 is positively retained onto the syringe 61. This arrangement is known as a “Luer Lock”.

According to a third embodiment of the invention the longitudinal passage of the first and second embodiments is of a constant diameter between the sub-chamber and the second socket 25. However the diameter of the longitudinal passage is greater than the diameter of the needle 15.

According to an alternative embodiment of the invention the longitudinal passage of the first and second embodiments is of a tapered diameter which is convergent towards the second socket 25. However the diameter of the longitudinal passage in the region of its intersection with the bore 27 greater than the diameter of the needle 15.

The present invention is not to be limited in scope by any of the specific embodiments described herein. These embodiments are intended for the purpose of exemplification only. Functionally equivalent products, formulations and methods are clearly within the scope of the invention as described herein.

1. An attachment for a parenteral device wherein the parenteral device comprises a body defining space and a duct having an opening at its outer end, the duct in use is intended to convey parenteral fluid to or from the space, said attachment comprising a housing adapted to be removably engageable with the body and to be supported by the body, the housing having an engagement portion having a flow path which is to be in communication with the opening and the duct when the engagement portion is in said sealing engagement with the body the housing further comprising an elongate portion slidably supporting a hollow needle wherein the needle has a free end which is open and a handle mounted to the other end, the other, end of the needle being closed, the needle provided with a second opening formed through the wall of the needle intermediate of the ends of the needle, the needle being sidably accommodated within the elongate housing to be movable from or to a stored position at which the free end is sealingly accommodated within the housing to or from an extended position at which the free end extends beyond the housing, the needle being further movable to a retraced position at which the free end is located in a non-sealing manner within the housing, wherein the one end of the housing is formed with a bore through which the needle is slidably and sealingly received, wherein a sub-chamber which is open to the flow path is located intermediate of the length of the bore, wherein the sub-chamber is located such that the second opening is located within the sub-chamber on the needle being moved to the extended position, the dimensions of the sub-chamber enabling open communication between the interior of the needle and the sub-chamber when the opening is located within the sub-chamber irrespective of the angular orientation of the needle about the longitudinal axis of the needle.

2. An attachment for a parenteral device as claimed at claim 1 wherein the free end is located within the bore but outward of the sub-chamber when the needle is in the stored position and the free end is located inward of the bore when the needle is located in the retracted position.

3. An attachment for a parenteral device as claimed at claim 1 wherein the duct comprises a tubular spigot extending from the parental device.

4. An attachment for a parenteral device as claimed at claim 6 wherein the spigot has a configuration of a “Luer” fitting.

5. An attachment for a parenteral device as claimed at claim 7 wherein the “Luer” fitting is associated with a “Luer Lock”

6. An attachment for a parenteral device as claimed at claim 1 wherein the engagement portion includes a socket which is adapted to sealingly engage the body to surround the opening, said socket being located to one side of the elongate portion, said flow path extending between the socket and the sub-chamber.

7. An attachment for a parenteral device as claimed at claim 9 wherein the flow path comprises a first longitudinal passage formed through the engagement portion said longitudinal passage connecting the socket with the sub-chamber.

8. An attachment for a parental device as claimed at claim 10 wherein the sub chamber is a part of the longitudinal passage and is coaxial therewith and the longitudinal passage intersects the bore in the region of the sub-chamber.

9. An attachment for a parenteral device as claimed at claim 11 wherein the sub chamber has a diameter greater than the longitudinal passage and a diameter greater than the diameter of the bore.

10. An attachment for a parenteral device as claimed at claim 11 wherein the sub chamber has a diameter substantially equal to the diameter of the longitudinal passage.

11. An attachment for a parenteral device as claimed at claim 11 wherein the longitudinal passage including the sub chamber has tapered internal cross-section which is convergent in the direction away from the bore.

12. An attachment for a parenteral device as claimed at claim 11 wherein the diameter of the sub-chamber is greater than the diameter of the bore.

13. An attachment for a parenteral device as claimed at any one of claims 11 to 15 wherein the longitudinal passage is substantially perpendicular to the bore.

14. An attachment for a parenteral device as claimed at claim 10 wherein the longitudinal passage opens to the exterior of the engagement portion in opposed relation to the socket and the outer end of the longitudinal passage is closed.

15. An attachment for a parenteral device as claimed at claim 11 wherein the longitudinal passage is closed subsequent to the formation of the longitudinal passage.

16. An attachment for a parenteral device as claimed at claim 1 wherein the engagement portion is formed as a separate element from the elongate portion.

17. An attachment for a parenteral device as claimed at claim 1 wherein the handle comprises a first element which is slidably received in the elongate body and which closes the other end of the needle, the first element supporting a protuberance which extends from the elongate body and is engagable by an operator to effect said slidable movement of the needle.

18. An attachment for a parenteral device as claimed at claim 20 wherein the handle supports a pawl which is engagable with one of a set of longitudinally spaced recesses in the elongate portion to retain the needle in the stored, extended and retracted position.

19. An attachment for a parenteral device as claimed at claim 21 wherein the pawl is resiliently supported from the first element such that it is biased laterally with respect to the direction of movement of the needle to become engaged with one of said recesses on the pawl moving into alignment with a recess.

20. An attachment for a parenteral device as claimed at claim 22 wherein an increased biasing is applied to the pawl when in engagement with the recess at which the needle is in the retracted position.

21. An attachment for a parenteral device as claimed at claim 1 wherein the parenteral device comprises a syringe and wherein when the syringe has the attachment engaged therewith the central axes of the elongate potion and the syringe are substantially parallel and the elongate portion lies closely adjacent to the syringe.

22. (canceled)
